(19) United States
(12) Patent Application Publication
(10) Pub. No.: US 2025/0090046 A1
(43) Pub. Date: Mar. 20, 2025

(54) PERFORMANCE FORECASTING FOR ACTIVITIES

(71) Applicant: Apple Inc., Cupertino, CA (US)

(72) Inventors: Paul G. Puskarich, Palo Alto, CA (US); Fiona P. O`Leary, Palo Alto, CA (US)

(21) Appl. No.: 18/370,857

(22) Filed: Sep. 20, 2023

(51) Int. Cl. A61B 5/11; A61B 5/00; A61B 5/01; A61B 5/024; A61B 5/0533; A61B 5/08

(52) U.S. Cl. CPC A61B 5/1118; A61B 5/01; A61B 5/024; A61B 5/0533; A61B 5/0816; A61B 5/6802

(57) ABSTRACT

Embodiments are disclosed for performance forecasting for activities. In some embodiments, a method comprises: obtaining personal prior assessment information associated with a user, obtaining historical performance information associated with a previous activity, obtaining activity assessment information for a current activity; and forecasting a performance of the current activity by the user based on the personal prior assessment information, the historical performance information, and the activity assessment information.

### TECHNICAL FIELD

This disclosure relates generally to health monitoring and fitness applications.

### BACKGROUND

Many existing mobile and wearable devices, such as smartphones, smartwatches, and earbuds include sensors that provide data that can be used to determine the health and/or fitness of a user. These devices may include a fitness application that allows a user to select a route for walking, running, biking, or hiking. For example, a user may query a hiking application to find the best trails near the user and provide reviews from other users to find out about current trail conditions.

Sensor data, such as a heart rate and motion data can be used to track the user's vitals and movements during physical activities from which health and performance metrics can be computed, including but not limited to heart rate, respiratory rate, caloric expenditure, recovery rate, etc. Some metrics can provide an indication of the user's ability to engage in physical activities. For example, some mobile devices, such as Apple Inc.'s iPhone® provide mobility metrics, such as estimates of walking speed, step length, double support time, and walking asymmetry. These mobility metrics can be used to characterize the user's gait and mobility.

A problem often encountered by users who wish to engage in a physical activity, such as hiking, is their inability to assess their performance of the activity. For example, a user may not know if they can handle a particular hiking trail without overexerting themselves. There are many variables that can impact their performance, such as the user's physical fitness, injuries, trail conditions, trail difficulty, weather, etc. Overexertion while performing an activity could result in the user feeling dizzy, sore, hot, sweaty, or have a high pulse rate, abdominal pain, heart flutters or chest pain.

### SUMMARY

Embodiments are disclosed for performance forecasting for activities. In some embodiments, a method comprises: obtaining personal prior assessment information associated with a user, obtaining historical performance information associated with a previous activity associated with the user or other users, obtaining activity assessment information for a current activity; and forecasting a performance of the current activity by the user based on the personal prior assessment information, the historical performance information, and the activity information.

In some embodiments, the personal prior assessment information includes health or fitness information computed from sensor data provided by one or more sensors of the mobile or wearable device.

In some embodiments, the personal prior assessment information is obtained from user input through a user interface of the mobile or wearable device.

In some embodiments, the personal prior assessment information includes mental wellbeing information obtained from the user input.

In some embodiments, the user input includes a description of the user's age and one or more physical characteristics.

In some embodiments, the user input includes a description of at least one of apparel, footwear, mobility aids, or items worn or carried by the user.

In some embodiments, the historical performance information includes at least one of heart rate, respiratory rate, body temperature, galvanic skin response, muscle activity, or user survey data.

In some embodiments, the current activity is one of walking, running, jogging, cycling, strolling, or hiking.

In some embodiments, the activity assessment information includes at least one of route surface conditions, elevation, grade, weather conditions, sun exposure, vistas, availability of accommodations, or traffic density.

In some embodiments, the forecasted performance is based on at least one of a purpose of the activity, whether the activity was previously completed by the user or other users, or inferences drawn from similar activities performed by the user or other users.

In some embodiments, the forecasted current performance includes a description of how the user may feel during or after completion of the activity.

In some embodiments, a system comprises: at least one processor; memory storing instructions that when executed by the at least one processor, cause the at least one processor to perform any of the preceding methods.

In some embodiments, a non-transitory, computer-readable medium has instructions stored thereon, that when executed by at least one processor, cause the at least one processor to perform any of the preceding methods.

Other embodiments are directed to an apparatus, system and computer-readable medium.

Particular embodiments described herein provide one or more of the following advantages. Users are provided with more detailed information regarding a particular activity they intend to undertake based on their health, fitness level, past performance for the activity, crowdsourced performance data, weather conditions, activity information and any other information that can be used to forecast the user' performance of the activity prior to undertaking the activity. The performance forecast allows a user to make an informed decision regarding whether to participate in a particular activity that could potentially exacerbate a medical condition, result in discomfort, and/or result in over excerption.

### BRIEF DESCRIPTION OF THE DRAWINGS

FIG. 1 is a flow diagram illustrating performance forecasting for activities, according to one or more embodiments.

FIG. 2 is a block diagram of a system for performance forecasting for activities, according to one or more embodiments.

FIG. 3 is a flow diagram of a process of performance forecasting for activities, according to one or more embodiments.

FIG. 4 is a block diagram of a device architecture for implementing the features and processes described in reference to FIGS. 1-3.

### DETAILED DESCRIPTION

### Overview

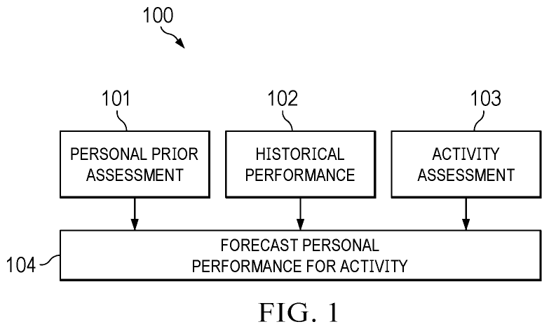

FIG. 1 is a flow diagram 100 illustrating performance forecasting for activities, according to one or more embodiments. To provide a performance forecast various health and fitness information is collected and stored on a mobile or wearable device, and/or on a network. For example, personal prior assessment information 101 can be stored in one or more databases and includes without limitation the user's age, gender, cardiovascular age, height, weight, surgeries (e.g., hip transplants, etc.) or other health impairments (e.g., pacemaker, etc.), preferences for types of activities (e.g., walking, running, cycling, etc.), mobility aids (e.g., knee brace, walking stick, walker, wheelchair, etc.), apparel and footwear (e.g., high heels, sneakers, boots, jeans, athletic wear, etc.), carrying items (e.g., backpack, suitcase, etc.), moving others (e.g., child carrier, stroller, etc.), or a pet (e.g., a dog, a pet breed, etc.), personal wellbeing, e.g., mental wellbeing as determined by the user logging their moods and emotions or by taking a standardized mental health assessment.

In some embodiments, prior personal assessment information 101 is entered by user input through a graphical user interface (GUI) of a mobile application running on the mobile/wearable device, such as a health or fitness application. In some embodiments, prior personal assessment information 101 can be obtained over time from the one or more mobile applications running on the mobile/wearable device. For example, a health application can compute various health metrics over time, such as heart rate, respiratory rate, mobility metrics, etc., based on sensor data provided by one or more sensors (e.g., heart rate sensor, motion sensors, etc.) of the mobile/wearable device.

In addition to personal prior assessment information 101, historical performance information 102 associated with an activity is obtained and stored in one or more databases. Historical performance information 102 includes past performance data (e.g., heart rate, respiratory rate, body temperature, galvanic skin response, etc.) associated with the activity (e.g., hiking, walking, cycling, strolling, running/jogging, skiing, swimming, etc.). For example, performance data can include the user's heart rate and/or respiratory rate associated with the activity (e.g., hiking on a trail or route, cycling on a trail or route, etc.), user-reported/rated survey data (e.g., reporting on comfort, exertion, enjoyment, etc.), and performance data (e.g., heart rate, respiratory rate, galvanic skin response, survey data, muscle activity, etc.) for other users who performed the same activity in the past, which can be obtained by, for example, crowdsourcing.

In addition to personal prior assessment information 101 and historical performance information 102, activity assessment information 103 information is obtained that is related to the activity the user intends to undertake. An activity can be any physical activity including hiking, walking, jogging, running, strolling, cycling, swimming, aerobics training, shopping, dancing, sight-seeing, participating in any sport (e.g., skiing, tennis, scuba diving, etc.), etc. The types of activity assessment information can depend on the activity. In an example hiking scenario, the user can invoke a hiking mobile application on their mobile/wearable device and initiate a search query for a hiking trail nearby. The user can enter various filters for the search query, such as outdoors (e.g., national park, bike trails, etc.), outdoors city (e.g., Venice, London, etc.), indoors (e.g., shopping mall, track, etc.), etc. The user can apply search filters for surface conditions (e.g., paved, cobblestone, boardwalk, gravel, elevation (e.g., flat, incline, steps, etc.), etc. In some embodiments, the user can enter the conditions that they observe at the trail, which can be crowdsourced and provided in search results to other users. An example hiking application is AllTrails® mobile application available on the World Wide Web at https://alltrails.com. All or some of the foregoing information are examples of activity assessment information 103 for hiking activities. Other activities could have additional or different assessment information.

In some embodiments, the user can also search for weather conditions (e.g., rainy, foggy, UV exposure, humidity, etc.) at the trail, and/or surface conditions due to the weather (e.g., wet, slippery, icy, dry, snow, etc.) on the hiking application or in a separate weather application.

In some embodiments, the user can filter for user traffic density on the trail (e.g., how many people are currently using the trail). The traffic density information can be based on crowd sourced information, for example.

Other miscellaneous assessment information 103 related to a hiking activity can be entered into the hiking application, such as whether the trail is narrow or wide, winding or straight, and whether there are accommodations (e.g., restroom, water fountains, benches for resting, vistas or viewpoints, etc.), etc. Based on the search filters selected by the user, a list of proposed hiking trails is presented to the user on the GUI.

In some embodiments, the user's performance for the proposed activity is forecasted 104 based on personal prior assessment information 101, historical performance information 102 and activity assessment information 103. The user's forecasted performance 104 for the activity can include data or metrics that indicate whether the user can complete the activity safely and comfortably based on their personal prior assessment information 101 (e.g., medical conditions) and historical performance information 102 for the proposed activity. The performance forecast can be influenced by additional information such as the purpose of the activity. For example, the purpose can be sightseeing, exercise, daily commute, miscellaneous errands, etc.

In some embodiments, performance information crowdsourced from other users having similar prior personal assessment information 101 can be used to influence the performance forecast. For example, other users may have reported to the hiking app that they were exhausted or sweating during the hike, advise that certain equipment/supplies is needed for the hike (e.g., wear hiking boots, wear hat, bring water, bring sunscreen, bring insect repellent, etc.).

Some examples of a performance forecast include providing a qualification of how the user will feel after the activity, e.g., comfortable, tired/sweating, over-exerted (need to rest). In some embodiments, the performance forecast can qualify whether the user is suited for the activity based on their personal prior assessment information 101, historical performance information 102 and activity assessment information 103 (e.g., search results for a proposed hiking trail). If not suited, the application can warn the user to not perform the activity, offer an alternative activity at the same location or other location (e.g., nearby location), or provide any other guidance to the user (e.g., consult your doctor first, carry water or food).

In some embodiments, the performance forecast can be in the form of a text description and/or a metric. For example, the user can be given a performance score for the proposed route, where the score is personalized to the user. For example, a performance score can be computed based on a sum of weighted factors, the user's reported/monitored physical condition, the user's reported/monitored injuries, the user's historical performance for the activity (e.g., how the user performed in a previous hike on the same trail), route characteristics (e.g., paved, dirt, muddy, slippery, etc.), the weather conditions (e.g., rainy, hot, foggy, snow, etc.) and other information, such as the user's attire (e.g., whether wearing appropriate hiking shoes, hat, sun protection, etc.), purpose for the activity (e.g., sightseeing, exercise, etc.), the presence of accommodations (e.g., wheelchair access, drinking fountains, restrooms, first aid or rangers stations, etc.), etc. The weights applied to the factors can be based on the impact on the user's performance of the activity. For example, a reported injury could be weighted more than the weather conditions because it may have a greater impact on the user's performance of the activity. In some embodiments, the weights can be a number between 0.0 and 1.0. In some embodiments, the performance score can be used to index a database of textual and/or visual information which is presented on the GUI of their mobile/wearable device. In some embodiments, the prediction score can be converted to a probability that the user will be comfortable (e.g., pain free, within an energy expenditure range, etc.) during their hike.

In some embodiments, supervised or unsupervised machine learning can be used to generate a performance forecast. For example, a neural network can extract feature vectors from the personal prior assessment information 101, historical performance information 102 and activity assessment data 103 and input the feature vectors into a multi-layer convolutional neural network or other deep learning network which can be trained on crowdsourced data. The output of the neural network can be a performance score for the activity. In some embodiments, the performance score can qualify whether the user is suited for the activity.

In some embodiments, a recommendation engine can be used to recommend a route for an activity to the user based on personal prior assessment information 101, historical performance information 102 and activity assessment information 103. Route as used herein refers to hiking trails, walking paths, bike paths, etc. In some embodiments, the recommendation engine can be implemented using collaborative filtering (e.g., such as singular value decomposition (SVD)) and/or content-based filtering). In some embodiments, crowdsourced fitness scores can be used as input into a recommendation engine. The recommendation engine could be used to qualify multiple proposed routes that are provided in a search result. The recommendation engine could look at the users predicted performance score and use the performance score to match the user with a particular route from the multiple proposed routes.

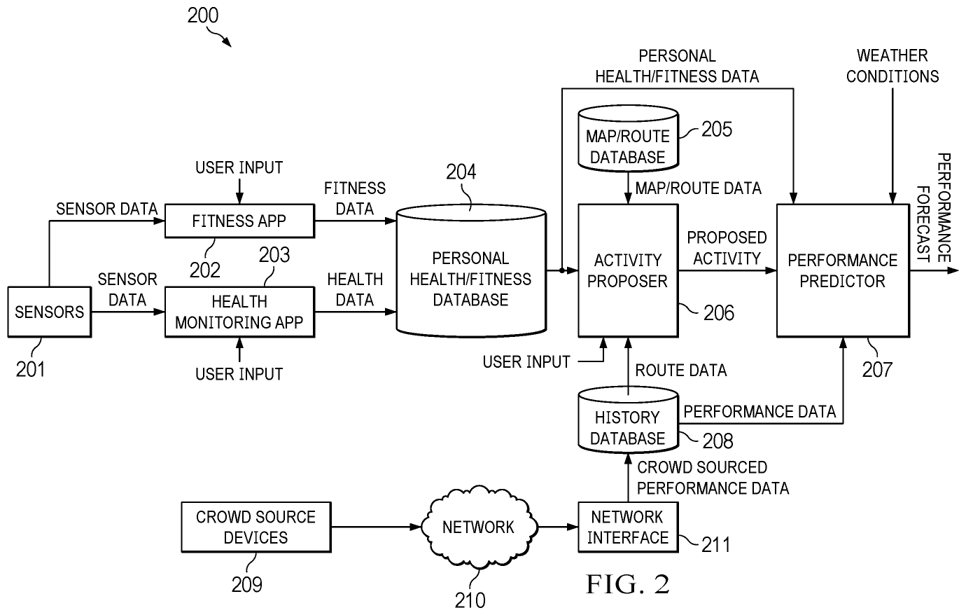

FIG. 2 is a block diagram of a system 200 for fitness forecasting for activities, according to one or more embodiments. System 200 includes personal health/fitness database 204, activity proposer 206, map/route database 205, activity history database 208 and performance predictor 207.

Sensor data 201 output by one or more sensors of a mobile/wearable device is ingested by fitness application 202 and health monitoring application 203 which use sensor data 201 to automatically compute fitness and health data and/or metrics. For example, health monitoring application 203 can compute a mobility metric for the user as an indicator of a user's injury, disability, and short- and long-term health. An example of a health monitoring application is Apple Inc.'s Health application. A user's walking mobility is affected by a variety of health conditions including muscular degeneration, neurological disease, and cardiopulmonary fitness. Some examples of mobility metrics include but are not limited to walking speed, step length, double support time, and walking asymmetry. Walking mobility metrics are described more fully in the publication, “Measuring Walking Quality Through Mobility Metrics,” May 2022 (https://www.apple.com/healthcare/docs/site/Measuring_Walking_Quality_Through_iPhone_Mobility_Metrics.pdf).

Some examples of fitness metrics include but are not limited to heart rate, stride length, ground contact time, vertical oscillation, average power, and VO₂ max. An example fitness application 202 is Apple Inc.'s Workout app which provides an estimate of a user's cardio fitness level (VO₂ max estimate) using heart rate and motion sensors in the Apple Watch® during an outdoor walk, outdoor run, or hiking workout. To estimate the user's cardio fitness level, the Workout app accounts for the user's age, sex, weight, height, and any medications that might affect the user's heart rate. The mobility metrics and fitness metrics can be computed during throughout the day and stored in personal health/fitness database 204, which can be located on the mobile/wearable device and/or on a network storage device where it can be viewed from other devices (e.g., a desktop computer).

Activity proposer 206 proposes an activity for a user, such as hiking, biking, walking, running, jogging, sightseeing, skiing, tennis, swimming, etc. In some embodiments, the activity is proposed using personal prior information 101 stored in personal health/fitness database 204, along with map/route data from map/route database 205 (if the activity requires a route) and activity history data from activity history database 206. In some embodiments, a single database can be used. Map-route data includes the map data (e.g., polylines with metadata) needed to generate routes based on user input, such as a filtered search query. Activity proposer 206 can include a search engine for performing a search or receive the results of a search. Activity proposer 206 can be implemented in a mobile application (e.g., a hiking application), as a network or operating system service, or any other suitable manner.

The output of the activity proposer 206 is a list of one or more proposed activities based on the user's search query. If information from personal health/fitness database 204 is used to filter the search, then the proposed activities will account for the user's health and fitness information. For example, if the user has a low fitness score or mobility score, the proposed activity will require less physical exertion (e.g., shorter distance, less inclines, paved surfaces, etc.).

In some embodiments, activity history data from database 208 is also used by activity proposer 206 to select one or more previous activities associated with the user in the past. The activity history data can include performance data (e.g., a performance score) output by performance predictor 207. In some embodiments, activity history data can include crowd sourced data, such as, for example, an average performance score based on performance scores achieved by other users who have engaged in the activity and have similar health and fitness metrics. In some embodiments, the similarity can be determined using a similarity metric (e.g., a distance metric, trail, road, etc.) and/or a classifier (e.g., k-nearest neighbor (KNN) algorithm). The crowd sourced data can be received from crowd source devices 209 (e.g., smartphones, smartwatches, fitness bands implementing system 200) through network 210 (e.g., the Internet) and network interface 211 (e.g., a database manager).

Performance predictor 207 computes prediction data (also referred to herein as a “performance forecast”) that can be presented to the user on, for example, a GUI of their mobile/wearable device prior to undertaking an activity associated with the route. In some embodiments, performance predictor 207 computes a performance score based on personal health/fitness data, activity data and weather conditions (e.g., provided by a weather application). Activity data can include but is not limited to any information related to the activity to be undertaken by the user. For example, if the activity involves a route, then activity data could include route length, elevation, surface conditions, number of turns, inclines, availability of accommodations (e.g., restrooms, wheel-chair access, water founts).

As previously disclosed, all of the foregoing information can be included in a weighted some of factors to compute a performance score, which can be presented to the user on a GUI with text giving the reasons for the score, warnings, recommendations for items to bring (e.g., bring water and a hat), etc. In some embodiments, the performance score can be used to filter out proposed activities for the user automatically or in response to a user selected option in a mobile application.

The foregoing will now be illustrated by example. Bob decides he wants to go for a hike. He takes a trip to a national park and discovers that there many different trails to hike. He uses a hiking application on his smartwatch to determine which route to take. He uses a filter that requests a scenic route that is between 5-10 miles in length. He also indicates through input into the GUI that he is not wearing hiking shoes.

The hiking application produces a number of trails based on Bob's search criteria. In some embodiments, Bob's personal prior assessment information is also used to search for trails that are suitable for Bob based on the personal prior assessment information 101 stored in personal health/fitness database 204. In some embodiments, the personal prior assessment information 101 includes a cardio fitness level score computed by a health app running on his smartwatch and also a mobility metric computed by a health monitoring app running on his smartwatch. Bob does not exercise very often and has a lower-than-average fitness score for his age, gender, height, and weight. Bob is also on high blood pressure medication. Bob also has a left leg injury that causes him to favor his right leg. The hiking application also looks at stored history performance information 102 stored in activity history database 208 to determine if Bob has hiked any of the trails in the national park in the past, or average performance scores for other hikers who hiked the trails the past. In this example, Bob has not visited the national park below, but there is an average performance metric based on other users who have similar personal prior assessment information 101 (e.g., same age, gender, weight, height, high blood pressure, no hiking shows, low fitness, and leg injury) based on a similarity metric and/or classifier algorithm.

In addition to the prior assessment information 101, the hiking application requests and obtains local weather conditions, which indicate the temperature is 85 degrees and sunny. All of this information is used to generate a list of proposed hiking trails at the national park, and performance forecast that indicates how Bob will likely perform on the proposed hiking trails.

In this example, the list of proposed hiking trails include shorter, paved paths to accommodate Bob's lower cardio fitness level score and mobility metric due to the leg injury, as well as his lack of good hiking shoes and high blood pressure.

Accordingly, system 200 is advantageous over conventional applications by providing Bob with more information that Bob can used to select a hiking trail that will be safe and comfortable for Bob based on his physical fitness and any health issue he may have.

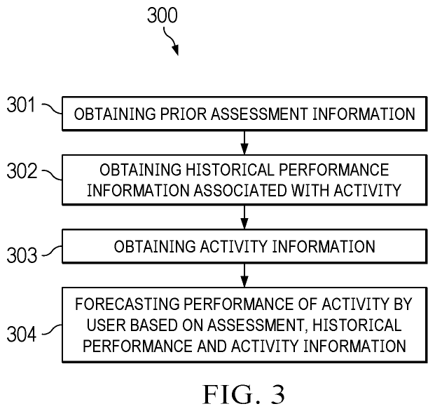

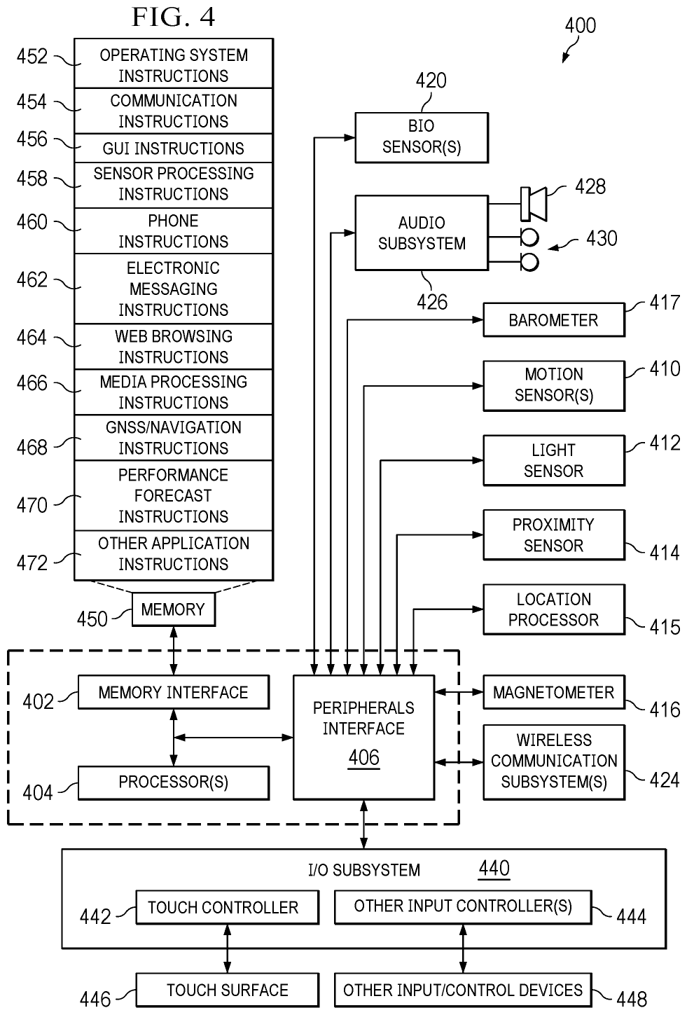

FIG. 3 is a flow diagram of a process 300 for fitness forecasting for activities, according to one or more embodiments. Process 300 can be implemented using, for example, the device architecture 400 described in reference to FIG. 4.

Process 300 includes obtaining assessment information associated with a user (301), obtaining historical performance information associated with a previous activity associated with the user or other users (302), obtaining activity information for a current activity (303); and forecasting a performance of the current activity by the user based on the assessment information, the historical performance information, and the activity assessment information (304). Each of these steps were previously described in response to FIGS. 1 and 2.

### Example Device Architecture

FIG. 4 is a block diagram of a device architecture 400 for implementing the features and processes described in reference to FIGS. 1-3. Architecture 400 can include memory interface 402, one or more hardware data processors, image processors and/or processors 404 and peripherals interface 406. Memory interface 402, one or more processors 404 and/or peripherals interface 406 can be separate components or can be integrated in one or more integrated circuits. System architecture 400 can be included in any suitable electronic device for crash detection, including but not limited to: a smartwatch, smartphone, fitness band and any other device that can be attached, worn, or held by a user.

Sensors, devices, and subsystems can be coupled to peripherals interface 406 to provide multiple functionalities. For example, one or more motion sensors 410, light sensor 412 and proximity sensor 414 can be coupled to peripherals interface 406 to facilitate motion sensing (e.g., acceleration, rotation rates), lighting and proximity functions of the wearable device. Location processor 415 can be connected to peripherals interface 406 to provide geo-positioning. In some implementations, location processor 415 can be a GNSS receiver, such as the Global Positioning System (GPS) receiver. Electronic magnetometer 416 (e.g., an integrated circuit chip) can also be connected to peripherals interface 406 to provide data that can be used to determine the direction of magnetic North. Electronic magnetometer 416 can provide data to an electronic compass application. Motion sensor(s) 410 can include one or more accelerometers and/or gyros configured to determine change of speed and direction of movement. Barometer 417 can be configured to measure atmospheric pressure (e.g., pressure change inside a vehicle). Bio signal sensor 420 can be one or more of a PPG sensor, an electroencephalogram (EEG) sensor, an electrocardiogram (ECG) sensor, an electromyogram (EMG) sensor, a mechanomyogram (MMG) sensor (e.g., piezo resistive sensor) for measuring muscle activity/contractions, an electrooculography (EOG) sensor, a galvanic skin response (GSR) sensor, a magnetoencephalogram (MEG) sensor and/or other suitable sensor(s) configured to measure bio signals.

Communication functions can be facilitated through wireless communication subsystems 424, which can include radio frequency (RF) receivers and transmitters (or transceivers) and/or optical (e.g., infrared) receivers and transmitters. The specific design and implementation of the communication subsystem 424 can depend on the communication network(s) over which a mobile device is intended to operate. For example, architecture 400 can include communication subsystems 424 designed to operate over a GSM network, a GPRS network, an EDGE network, a WiFi™ network and a Bluetooth™ network. In particular, the wireless communication subsystems 424 can include hosting protocols, such that the crash device can be configured as a base station for other wireless devices.

Audio subsystem 426 can be coupled to a speaker 428 and a microphone 30 to facilitate voice-enabled functions, such as voice recognition, voice replication, digital recording and telephony functions. Audio subsystem 426 can be configured to receive voice commands from the user.

I/O subsystem 440 can include touch surface controller 442 and/or other input controller(s) 444. Touch surface controller 442 can be coupled to a touch surface 446. Touch surface 446 and touch surface controller 442 can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with touch surface 446. Touch surface 446 can include, for example, a touch screen or the digital crown of a smart watch. I/O subsystem 440 can include a haptic engine or device for providing haptic feedback (e.g., vibration) in response to commands from processor 404. In an embodiment, touch surface 446 can be a pressure-sensitive surface.

Other input controller(s) 444 can be coupled to other input/control devices 448, such as one or more buttons, rocker switches, thumbwheel, infrared port, and USB port. The one or more buttons (not shown) can include an up/down button for volume control of speaker 428 and/or microphone 430. Touch surface 446 or other controllers 444 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 446; and a pressing of the button for a second duration that is longer than the first duration may turn power to the mobile device on or off. The user may be able to customize a functionality of one or more of the buttons. The touch surface 446 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 402 can be coupled to memory 450. Memory 450 can include high-speed random-access memory and/or non-volatile memory, such as one or more magnetic disk storage devices, one or more optical storage devices and/or flash memory (e.g., NAND, NOR). Memory 450 can store operating system 452, such as the iOS operating system developed by Apple Inc. of Cupertino, California. Operating system 452 may include instructions for handling basic system services and for performing hardware dependent tasks. In some implementations, operating system 452 can include a kernel (e.g., UNIX kernel).

Memory 450 may also store communication instructions 454 to facilitate communicating with one or more additional devices, one or more computers and/or one or more servers, such as, for example, instructions for implementing a software stack for wired or wireless communications with other devices. Memory 450 may include graphical user interface instructions 456 to facilitate graphic user interface processing; sensor processing instructions 458 to facilitate sensor-related processing and functions; phone instructions 460 to facilitate phone-related processes and functions; electronic messaging instructions 462 to facilitate electronic-messaging related processes and functions; web browsing instructions 464 to facilitate web browsing-related processes and functions; media processing instructions 466 to facilitate media processing-related processes and functions; GNSS/Location instructions 468 to facilitate generic GNSS and location-related processes and instructions; and instructions 470 that implement the processes described in reference to FIGS. 1-3. Memory 450 further includes other application instructions 472 including but not limited to instructions for applications that implement system 200 shown in FIG. 2 (e.g., hiking applications, biking applications, running/jogging applications).

Each of the above identified instructions and applications can correspond to a set of instructions for performing one or more functions described above. These instructions need not be implemented as separate software programs, procedures, or modules. Memory 450 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
obtaining, with at least one processor of a mobile or wearable device, personal prior assessment information associated with a user;
obtaining, with the at least one processor, historical performance information for a previous activity associated with the user or other users;
obtaining, with the at least one processor, activity assessment information associated with a current activity; and
forecasting, with the at least one processor, a performance of the current activity by the user based on the personal prior assessment information, the historical performance information, and the activity assessment information.

2. The method of claim 1, wherein the personal prior assessment information includes health or fitness information computed from sensor data provided by one or more sensors of the mobile or wearable device.

3. The method of claim 1, wherein the personal prior assessment information is obtained from user input through a user interface of the mobile or wearable device.

4. The method of claim 1, wherein the personal prior assessment information includes mental wellbeing information obtained from user input.

5. The method of claim 3, wherein the personal prior assessment information includes a description of the user's age and one or more physical characteristics of the user.

6. The method of claim 3, wherein the personal prior assessment information includes a description of at least one of apparel, footwear, mobility aids, or items worn or carried by the user.

7. The method of claim 1, wherein the historical performance information includes at least one of heart rate, respiratory rate, body temperature, galvanic skin response, muscle activity, or user survey data.

8. The method of claim 1, wherein the current activity is one of walking, running, jogging, cycling, strolling, or hiking.

9. The method of claim 1, wherein the activity assessment information includes at least one of route or trail surface conditions, elevation, grade, weather conditions, sun exposure, vistas, availability of accommodations, or traffic density.

10. The method of claim 1, wherein the forecasted performance is based on at least one of a purpose of the current activity, whether the current activity was previously completed by the user or other users, or inferences drawn from similar activities performed by the user or other users.

11. The method of claim 10, wherein the forecasted performance includes a description of how the user may feel during or after completion of the current activity.

12. A system comprising:
at least one processor;
memory storing instructions that when executed by the at least one processor, cause the at least one processor to perform operations comprising:
obtaining personal prior assessment information associated with a user;
obtaining historical performance information for a previous activity associated with the user or other users;
obtaining activity assessment information associated with a current activity; and
forecasting a performance of the current activity by the user based on the personal prior assessment information, the historical performance information, and the activity assessment information.

13. The system of claim 12, wherein the personal prior assessment information includes health or fitness information computed from sensor data provided by one or more sensors of the mobile or wearable device.

14. The system of claim 12, wherein the personal prior assessment information is obtained from the user input through a user interface of the mobile or wearable device.

15. The system of claim 12, wherein the personal prior assessment information includes mental wellbeing information.

16. The system of claim 12, wherein the personal prior assessment information includes a description of the user's age and one or more physical characteristics of the user.

17. The system of claim 12, wherein the personal prior assessment information includes a description of at least one of apparel, footwear, mobility aids, or items worn or carried by the user.

18. The system of claim 12, wherein the historical performance information includes at least one of heart rate, respiratory rate, body temperature, galvanic skin response, muscle activity, or user survey data.

19. The system of claim 12, wherein the current activity is one of walking, running, jogging, cycling, strolling, or hiking.

20. The system of claim 12, wherein the activity assessment information includes at least one of route or trail surface conditions, elevation, grade, weather conditions, sun exposure, vistas, availability of accommodations, or traffic density.
